(19) United States
(12) Patent Application Publication
(10) Pub. No.: US 2018/0297925 A1
(43) Pub. Date: Oct. 18, 2018

(54) METHODS FOR TOTAL SYNTHESIS OF RESOLVIN E1

(71) Applicant: Salzman Lovelace Investments, Ltd., Herzliya (IL)

(72) Inventor: Prakash Jagtap, North Andover, MA (US)

(21) Appl. No.: 15/767,926

(22) PCT Filed: Oct. 9, 2016
(86) PCT No.: PCT/IL2016/051095

§ 371 Date: Apr. 12, 2018

Related U.S. Application Data
(60) Provisional application No. 62/308,322, filed on Mar. 15, 2016.

(30) Foreign Application Priority Data
Oct. 12, 2015 (GB) 1518043.3

(51) Int. Cl. C07C 51/09; C07C 67/327; C07D 317/16; C07F 7/18; C07C 67/317; C07D 317/30; C07C 69/675; C07C 69/708; C07C 43/12; C07C 47/198; C07C 217/46; C07C 69/732

(52) U.S. Cl. CPC C07C 51/09; C07C 67/327; C07D 317/16; C07F 7/1892; C07C 67/317; C07D 317/30; C07C 69/732; C07F 7/1852; C07C 69/675; C07C 69/708; C07C 43/126; C07C 47/198; C07C 217/46; C07F 7/1856

(57) ABSTRACT

Methods for total chemical synthesis of Resolvin E1 (RvE1) include Wittig reaction of two compounds having hydroxyl protecting group in the presence of a strong base, removal of the hydroxyl-protecting groups with a deprotecting reagent to produce a compound having an ester group, and hydrolysis of the ester group to obtain RvE1

### TECHNICAL FIELD

The present invention provides methods for total chemical synthesis of Resolvin E1 (RvE1).

Abbreviations: ACN, acetonitrile; BAIB, bisacetoxyiodobenzene; CSA, camphorsulfonic acid; DCM, dichloromethane; DIBAL/DIBAL-H, diisobutylaluminum hydride; DIPEA, N,N-diisopropylethylamine; DMAP, 4-dimethylaminopyridine; DMF, dimethylformamide; EA, ethyl acetate; HMPA, hexamethylphosphoramide; Im, imidazole; KHMDS, potassium bis(trimethylsilyl)amide (potassium hexamethyldisilazane); LDA, lithium diisopropylamide; NaHMDS, sodium bis(trimethylsilyl)amide; PCC, pyridinium chlorochromate; PG, protecting group; p-Tosyl, p-toluenesulfonyl; p-TSA, p-toluenesulfonic acid; py, pyridine; rt, room temperature; RvE1, Resolvin E1; TBAF, tetra-n-butylammonium fluoride; TBDMS, tert-butyldimethylsilyl; TBDMSCl, tert-butyldimethylsilyl chloride; TBDPS, tert-butyldiphenylsilyl; TBDPSCl, tert-butyldiphenylsilyl chloride; TBS, tert-butyldimethylsilyl; TBSCl, tert-butschemeyldimethylsilyl chloride; TEA, triethylamine; TEMPO, 2,2,6,6-tetramethylpiperidin-1-yl)oxyl; THF, tetrahydrofuran; TMS, trimethylsilyl.

### BACKGROUND ART

Resolvin E1 (RvE1; 5(S),12(R),18(R)-trihydroxy-6Z,8E,10E,14Z,16Z-eicosapentaenoic acid) is an oxidative metabolite of the omega-3 fatty acid eicosapentaenoic acid (EPA). RvE1 is an endogenous lipid mediator and has been identified in local inflammation during the healing stage. RvE1 reduces inflammation in several types of animal models including peritonitis and retinopathy, and blocks human neutrophil transendothelial cell migration.

Due to its limited availability in the natural sources, it is of great importance to design methods for synthesis of RvE1 so as to evaluate its pharmaceutical properties and potential as anti-inflammatory. Such methods may also enable designing RvE1 analogues.

Recent publications (Allard et al., Tetrahedron Letters 2011, 52, 2623-2626; Ogawa and Kobayashi, Tetrahedron Letters, 2009, 50(44), 6079-6082) describe total synthesis of RvE1; however, these methods are not applicable to commercial manufacture for pharmaceutical use.

### SUMMARY OF INVENTION

In one aspect, the present invention provides various synthetic routes for the preparation of RvE1.

The synthetic routes disclosed herein, including full chemical structures of all the compounds involved, are shown in the Appendix hereinafter, Schemes 1-19, wherein the various starting compounds, intermediates and products referred to are herein identified by the Arabic numbers 1-48, 51-56, 59, 61, 62, 64, 65, and 68-73. RvE1 (in the form of the sodium salt thereof) is identified herein as compound 30.

Some of the compounds/intermediates synthesized are known; however, some of them are novel. In another aspect, the present invention thus provides the novel compounds 7, 8, 10, 13, 14, 15, 19, 20, 21, 23, 28, 29, 38, 39, 40, 41, 42, 47, 54, 59, 61, 65, 68, 69, 70, 71, 72, and 73, which are useful as intermediates in the syntheses disclosed herein.

### DETAILED DESCRIPTION

In one aspect, the present invention provides methods, i.e., procedures, for total chemical synthesis of RvE1 (compound 30).

In one particular such aspect, the invention provides a method for the synthesis of RvE1 starting from compound 28, said method is carried out as depicted in Scheme 1 and comprises: (i) selective removal of the TBDPS protecting groups at positions C12 and C18 of compound 28 and reduction of the triple bond at positions 6-7 to an olefinic bond, thus resulting in compound 29; and (ii) deacetylation of the protected hydroxyl group at position C5 of compound 29, to obtain RvE1. In a particular non-limiting embodiment, the TBDPS protecting groups at positions C12 and C18 of compound 28 are removed by treatment with TBAF in THF; the triple bond at positions 6-7 of compound 28 is reduced by treatment with activated Zn; and the protected hydroxyl group at position C5 of compound 29 is deacetylated by treatment with NaOH.

Compound 28 used in the synthesis of RvE1 may be obtained, e.g., as depicted in Scheme 2, by reaction of compound 17 with compound 22, or by reaction of compound 16 with compound 23.

Alternatively, compound 28 may be obtained as depicted in Scheme 3, by reaction of compound 32 with compound 33 in the presence of Pd(PPh₃)₄, CuI and Et₂NH. As shown in this scheme, compound 32 may be obtained from compound 31 by reaction with CrCl₂ and CHI₃; and compound 31 may be obtained from compound 16 by reaction with Ph₃P═CHCHO, benzene or ACN.

Compounds 16 and 17 may be synthesized from compound 10, e.g., as depicted in Scheme 4. The procedure described in this scheme involves several reactions in which compound 13 is prepared from compound 10 and converted to compound 14, followed by its conversion to compound 15. Reaction of compound 15 with BAIB/TEMPO leads to compound 16 while reaction of compound 15 with PPh₃, Im, I₂, and then with NaHCO₃, ACN, PPh₃, leads to compound 17.

Compound 10 may be obtained from compound 1, e.g., as depicted in Scheme 5. The procedure described in this scheme involves several reactions in which compound 2 is prepared from compound 1 by reaction with TBSCl, DMAP, DCM; compound 2 is converted to compound 3 by reaction with TBDPSCl, DMAP, Im DCM; compound 3 is converted to compound 4 by reaction with camphor sulfonic acid, 1:1 DCM:MeOH; compound 4 is converted to compound 5 by reaction with Dess-Martin periodinate and DCM, or with BAIB/TEMPO; compound 5 is reacted with Ph₃P═CHCHO to yield compound 6, followed by its conversion with DIBAL-H/toluene to compound 7 which is converted to compound 8 with PPh₃, Im, I₂, and then NaHCO₃, ACN, PPh₃. Reaction of compound 8 with compound 9 under KHMDS, THF leads to compound 10.

Alternatively, compound 10 may be synthesized from compound 11 as depicted in Scheme 6. As described in this scheme, compound 11 is reacted with PPH₃, Im, I₂, and then NaHCO₃, ACN, PPh₃, and the resulting compound 12a is reacted with compound 6 in the presence of KHMDS, THF, thus obtaining compound 10.

Compounds 22 and 23 may be synthesized from compound 18, e.g., as depicted in Scheme 7. As described in this scheme, compound 18 is converted to compound 19 with TBDPSCl, Im, DCM, and compound 19 is then converted to compound 20 by reaction with n-BuLi, THF, ClCO(CH₂)₃CO₂Et. Alternatively, compound 18 is converted directly to compound 20 by reaction with AlCl₃, glutaric anhydride, and then EtI/DIPEA. Compound 20 is converted to compound 21 using Noyori catalyst, Me₂CHOH or alpine borane, THF. Compound 21 is then converted to compound 22 by reaction with Ac₂O, NEt₃, THF, and then CSA, MeOH, or with BAIB/TEMPO, ACN; or to compound 23 by reaction with Ac₂O, NEt₃, THF, and then CSA, MeOH; and PPh₃, Im, I₂, and then NaHCO₃, ACN, PPh₃.

Compound 33 used in the synthesis of compound 28 may be obtained, e.g., from compound 27, which may be synthesized starting from compound 24, e.g., as depicted in Scheme 8. As particularly described in this scheme, compound 24 is converted to compound 25 by reaction with 2,6-dioxo-tetrahydropyran and AlCl₃, compound 25 is converted to compound 26 by reaction with p-TSA, Me₂CHOH, and compound 26 is converted to compound 27 using Noyori catalyst, Me₂CHOH or TBA, THF.

As shown in Scheme 1, each one of the positions C12 and C18 of compound 28, used for the synthesis of RvE1 according to the method disclosed above, is protected with TBDPS group, which is then removed to obtain compound 29. Yet, it should be understood that while TBDPS is the particular protecting group exemplified herein, other hydroxyl-protecting groups such as TBDMS might be used as well.

The term “hydroxyl-protecting group” as used herein refers to a group capable of masking hydroxyl groups during chemical group transformations elsewhere in the molecule, i.e., to a group capable of replacing the hydrogen atom of a hydroxy group on a molecule that is stable and non-reactive to reaction conditions to which the protected molecule is to be exposed. Examples of hydroxyl-protecting groups include, without being limited to, groups that can be reacted with hydroxyl groups to form ethers, such as silyl ethers (e.g., trimethylsilyl (TMS), triethylsilyl (TES), tert-butyldimethylsilyl (TBDMS; TBS), tert-butyldiphenylsilyl (TBDPS), or phenyldimethylsilyl ethers); substituted methyl ethers (e.g., methoxymethyl (MOM), benzyloxymethyl (BOM), tetrahydropyranyl (THP)); substituted ethyl ethers; benzyl ethers and substituted benzyl ethers; esters (e.g., acetate, formate, chloroacetate); and carbonates. Preferred hydroxyl-protecting groups are TBDPS, TBDMS and TBS. The removal of such groups to obtain the non-protected hydroxyl is carried out by using a deprotecting reagent, e.g., an acid, or a fluoride such as NaF, TBAF, HF-Py, or HF-NEt₃, as known to any person skilled in the art of organic chemistry.

In another particular such aspect, the invention provides a method for the synthesis of RvE1 starting from compound 42, said method is carried out as depicted in Scheme 9 and comprises: (i) reduction of the ester group of compound 42 to obtain compound 48; (ii) Wittig reaction of the aldehyde 48 with compound 47 in the presence of a strong base to obtain an intermediate product; and (iii) removal of the hydroxyl-protecting groups at positions C12 and C18 of said intermediate product and deacetylation of the protected hydroxyl group at position C5 of said intermediate product, to obtain RvE1. In a particular non-limiting embodiment shown in this scheme, the reduction of the ester group of compound 42 is carried out with DIBAL-H; the Wittig reaction is carried out in the presence of KHMDS; the deprotecting reagent used for removal of the TBDPS groups is TBAF; and deacetylation of the protected hydroxyl group at position C5 of said intermediate product is carried out with NaOH.

Compound 47 used in the synthesis of RvE1 may be obtained, e.g., as depicted in Scheme 10. As described in this scheme, 2-deoxy D-ribose (compound 34) is converted to compound 44 by reaction with (i) Ph₃P═C—CO₂Et, THF; compound 44 is converted to compound 45 by reaction with (ii) H₂/Pd—C, EtOH, (iii) DMP, (iv) Ac₂O, py; compound 45 is converted to compound 46 by reaction with (v) TFA-water, (vi) Pb(OAc)₄, DCM; and compound 46 is then converted to compound 47 by reaction with (vii) NaBH₄, THF, (viii) PPh₃, iodine, (9) PPh₃, NaHCO₃.

As shown in Scheme 9, the hydroxyl groups at positions C6 and C12 of compound 42 are protected with TBDPS groups, and the hydroxyl group at position C5 of compound 47 is acetylated, wherein all these groups are then removed to obtain compound 30. Yet, it should be understood that while TBDPS and acetyl are the particular protecting groups exemplified herein, other hydroxyl-protecting groups might be used as well.

In yet another particular such aspect, the invention provides a method for the synthesis of RvE1 starting from compounds 43 and 46, said method is carried out as depicted in Scheme 11 and comprises: (i) Wittig reaction of compound 43 with compound 46 in the presence of a strong base to obtain an intermediate product; and (ii) removal of the hydroxyl-protecting groups at positions C12 and C18 of said intermediate product and deacetylation of the protected hydroxyl group at position C5 of said intermediate product, to obtain RvE1. In a particular non-limiting embodiment shown in this scheme, the Wittig reaction is carried out in the presence of KHMDS; the deprotecting reagent used for removal of the TBDPS groups is TBAF; and deacetylation of the protected hydroxyl group at position C5 of said intermediate product is carried out with NaOH.

Compound 46 may be obtained starting from 2-deoxy D-ribose (compound 34), e.g. as depicted in Scheme 10, and compound 43 may be synthesized starting from compound 37, e.g., as depicted in Scheme 12.

Scheme 12 shows a procedure for the synthesis of compounds 42 and 43 starting from compounds 37 and 38. The procedure involves a series of reactions in which compounds 39, 40 and 41 are obtained. Compound 43 is obtained from compound 42 by reaction with DIBAL-H, toluene; PPh₃, iodine; PPh₃, NaHCO₃, ACN.

Compound 37 may be synthesized starting from 2-deoxy D-ribose (compound 34), e.g., as depicted in Scheme 13. As described in this scheme, compound 34 is converted to compound 35 by reaction with (i) Ph₃P═CH—CO₂Et, THF, (ii) NaOEt, EtOH; compound 35 is then converted to compound 36 by reaction with (iii) MsCl, py, (iv) NaI, acetone; and compound 36 is converted to compound 37 by reaction with (v) Ac₂O, py.

Compound 38 may be synthesized starting from compound 1, e.g., as depicted in Scheme 14. As described in this scheme, compound 1 is converted to compound 5, which is reacted with Br⁻Ph₃⁺P—C—C≡ in the presence of KHMDS, THF to obtain compound 38.

As shown in Scheme 11, the hydroxyl groups at positions C6 and C12 of compound 43 are protected with TBDPS groups, and the hydroxyl group at position C5 of compound 46 is acetylated, wherein all these groups are then removed to obtain compound 30. Yet, it should be understood that while TBDPS and acetyl are the particular protecting group exemplified herein, other hydroxyl-protecting groups might be used as well.

In a further particular such aspect, the invention provides a method for the synthesis of RvE1 starting from compounds 72 and 61, wherein PG each independently is a hydroxyl-protecting group such as TBDMS or TBDPS, said method is carried out as depicted in Scheme 15 and comprises: (i) Wittig reaction of compound 72 with compound 61 in the presence of a strong base; (ii) removal of the hydroxyl-protecting groups with a deprotecting reagent to obtain compound 73; and (iii) hydrolysis of the ester group of compound 73, to obtain RvE1. In a particular non-limiting embodiment, compound 72 is protected with two TBDPS groups; compound 61 is protected with TBDMS group; the Wittig reaction is carried out in the presence of KHMDS; and the deprotecting reagent used for removal of the hydroxyl-protecting groups is TBAF.

Compound 72 used in the synthesis of RvE1 may be obtained, e.g., as depicted in Scheme 16, by (i) Wittig reaction of compound 54 and compound 70, wherein PG each independently is a hydroxyl-protecting group such as TBDMS and TBDPS, in the presence of a strong base to obtain compound 71; and (ii) removal of the amide group of compound 71 with a strong base to obtain compound 72. In a particular non-limiting such embodiment, compound 54 is protected with TBDPS and compound 70 is protected with TBDMS; the Wittig reaction is carried out in the presence of KHMDS; and the removal of the amide group of compound 71 is carried out with DIBAL-H. Alternatively, compound 72 may be obtained as depicted in Scheme 15, starting from compound 54 and compound 12b, which is, in fact, a starting material for compound 70.

Compound 54 used in the synthesis of compound 72 may be obtained, e.g., as depicted in Scheme 17, by Wittig reaction of compound 53, wherein PG is a hydroxyl-protecting group such as TBDMS and TBDPS, and Ph₃P═CHCHO. In a particular non-limiting such embodiment, compound 53 is protected with TBDPS.

Compound 70 used in the synthesis of compound 72 may be obtained, e.g., starting from compound 64 as depicted in Scheme 18, by (i) deprotection of the diol in the presence of a weak acid; (ii) protection of the hydroxyl groups to obtain compound 65, wherein PG each independently is a hydroxyl-protecting group such as TBDMS and

TBDPS; (iii) Dess-Martin oxidation of compound 65 with Dess-Martin periodinate to obtain aldehyde 68; (iv) double Wittig reaction of aldehyde 68 with Ph₃P═CHCHO and then with Ph₃P═CHCON(OMe)Me to obtain compound 69; and (v) conversion of the compound 69 to the triphenylphosphonium salt 70 with triphenylphosphine. In a particular non-limiting such embodiment, compound 64 is deprotected in the presence of AcOH—H₂O, and the hydroxyl groups of the deprotected intermediate are then protected with either TBDMS or TBDPS to obtain compound 65. Compound 65 is oxidized, and the aldehyde obtained is then subjected to a double Wittig reaction as described above to obtain compound 70.

Compound 61 used in the synthesis of RvE1 may be obtained, e.g., as depicted in Scheme 19, by (i) reduction and deprotection of compound 56, followed by tosylation and then iodination to obtain compound 59; and (ii) hydroxyl-protection of compound 59 followed by conversion to the triphenylphosphonium salt 61 with triphenylphosphine. In a particular non-limiting such embodiment, compound 59 is hydroxyl protected by either TBDMS or TBDPS.

The methods for the synthesis of RvE1 disclosed herein are novel and have fewer steps and better overall yield compared with those of the prior art. The methods disclosed are also safer since they avoid exothermic steps known from the prior art that would be explosive when scaled up.

The enantioselectivity of the stereocenters in the RvE1 obtained by the methods starting from compounds 28 or 42, or by the reaction of compounds 43 and 46, is either obtained by using the appropriate chiral starting materials or introduced by reducing the keto-group with Noyori Catalyst. The enantiomeric excess (ee), a measurement of purity used for chiral substances, is measured after making the Mosher esters of corresponding chiral hydroxyls. The cis olefins are made by using KHMDS as a base and at lower temperature (0-78° C.). The thermodynamically stable trans olefins are made by a standard rt or reflux conditions. They are identified by their (corresponding protons) coupling constants (J values). The enantioselectivity of the stereocenters in the RvE1 obtained by the method starting from the reaction of compounds 72 and 61 is obtained by using the appropriate chiral starting materials.

The procedure for the synthesis of RvE1 starting from the compounds 72 and 61 is longer than the other synthetic procedures disclosed herein; however, it does not make use of metal-based catalysts such as those used in the other methods, e.g., the ruthenium-based Noyori catalyst, palladium and chromium used for Sonogashira coupling, or butyl lithium, and it is therefore significantly more cost effective.

In another aspect, the present invention provides the novel compounds 7, 8, 10, 13, 14, 15, 19, 20, 21, 23, 28, 29, 38, 39, 40, 41, 42, 47, 54, 59, 61, 65, 68, 69, 70, 71, 72, and 73, which are useful as intermediates in the syntheses disclosed herein.

The present invention further provides methods for the preparation of the known compounds/intermediates 6, 16, 17 and 22.

The invention will be now illustrated by the following non-limiting Examples.

### EXAMPLES

### Example 1. Synthesis of Compound 10

Compound 10 was synthesized as depicted in Schemes 5 and 6, according to the following procedure.

### Synthesis of Compound 2

As shown in Scheme 5, to a solution of imidazole (1 eq), TBSCl (1 eq), and DMAP (0.05 eq) in 40 mL of DCM at 0-5° C. was added the diol 1 (3 g, 33 mmol). The reaction was stirred and allowed to warm to ambient temperature overnight. The reaction was quenched with ammonium chloride, the product extracted with DCM, and the organic layer washed with sodium bicarbonate and brine, and then dried over sodium sulfate and concentrated. The material (6.24 g) was carried forward as is. Not UV active, but visible with vanillin (R_{f}=0.5 in 10% ethyl acetate/hexane).

### Synthesis of Compound 3

Compound 2 (6.24 g, 31.5 mmol) was added to a solution of TBDPSCl (1 eq), imidazole (1 eq), and DMAP (0.05 eq) in DCM at 0-5° C. The reaction was stirred and warmed to ambient temperature overnight. The reaction was quenched with ammonium chloride, the product extracted with DCM, and the organic layer washed with sodium bicarbonate and brine, and then dried over sodium sulfate and concentrated. The product was purified by column chromatography. Product is UV active at 254 nm. Column eluted with 0-5% ethyl acetate/hexane to give 11.4 g of compound 3 (82% yield).

### Synthesis of Compound 4

The bis-silyl ether 3 (8.3 g, 18.7 mmol) was dissolved in 1:1 DCM:MeOH (50 mL) at rt. Camphorsulphonic acid (0.5 eq) was added to the reaction mixture. The reaction was stirred for 2 h at rt. Triethylamine (1.1 eq) was added to the reaction mixture to quench. The mixture was concentrated and purified by column chromatography. Product is UV active at 254 nm. Chromatography with 0-20% ethyl acetate/hexane gave 5.34 g of product (87% yield).

### Synthesis of Compound 5

The starting material (1.7 g, 1 eq) was dissolved in 20 mL DCM and TEMPO (0.1 eq) was added. To the stirring reaction mixture was added BAIB (1.2 eq). The reaction was followed by TLC and complete after 3 h. To the reaction mixture was added TEA (2 mL), which was then concentrated and purified by column chromatography (0-20% ethyl acetate/hex). 1.3 g of product was isolated (77% yield).

### Synthesis of Compound 6

The aldehyde (9.1 g, 27.9 mmol) and (triphenylphosphoranylidene)acetaldehyde (1 eq) were dissolved in 120 mL of chloroform. The reaction was stirred at ambient temperature for 1 h and then refluxed for 2 h. The reaction mixture was concentrated and purified by column chromatography to give 4.9 g of product (50% yield). 1 H NMR (CDCl₃, 400 MHz): δ 0.84 (t, 3H, J=8.0 Hz), 1.08 (s, 9H), 1.51 (m, 2H), 4.43 (m, 1H), 6.17 (dd, 1H, J=16.0, 8.0 Hz), 6.68 (dd, 1H J=14.0, 6.0 Hz), 7.37 (m, 6H), 7.40 (m, 4H), 9.46 (d, 1H, J=8.0 Hz).

### Preparation of Wittig Salt 12a

As shown in Scheme 6, triphenylphosphine (3.96 g, 15.1 mmol) and imidazole (1.02 g) were dissolved in THF:ACN (3:1 25 mL). The mixture was cooled with an ice/water bath and iodine (3.8 g, 15.1 mmol) was added in 4 portions with vigorous stirring over a 20 minute period. The resulting slurry was warmed to rt and then cooled in an ice water bath. (4R)-4-(2-hydroxyethyl)-2,2-dimethyl-1,3-dioxolane (2 g, 13.7 mmol) was added dropwise to the reaction mixture. The resulting mixture was stirred at ambient temperature overnight in the dark. The reaction was checked for completeness by TLC (15% ethyl acetate/hexane-UV active R_{f}=0.5). The mixture was concentrated, diluted with 5% sodium bicarbonate solution and extracted with hexane. The combined organic layer was dried, concentrated and purified by silica gel chromatography. The product was isolated as 2.8 g of a light brown oil (80% yield) and used for the preparation of salt. 1 H NMR (CDCl₃, 400 MHz): δ 1.40 (s, 3H), 1.42 (s, 3H), 2.09 (m, 2H), 3.23 (m, 2H), 3.57 (dd, 1H, J=6.0, 6.0 Hz), 4.08 (dd, 1H J=6.0, 6.0 Hz), 4.15 (m, 1H).

A mixture of the iodo compound (1 g, 3.9 mmol), sodium bicarbonate (1 eq) and triphenylphosphine (1.2 g, 4.7 mmol) in 6 mL acetonitrile was stirred at 45 degrees (oil bath temperature) for 72 h with the flask covered by aluminum foil. The mixture was cooled to room temperature and filtered through a small pad of silica gel. The filter cake was washed with DCM and the filtrate concentrated. The residue was diluted with ether precipitating a white solid. The solid was filtered, rinsed with ether and dried under vacuum to afford the salt 12a. 550 mg, 30% yield. 1 H NMR (CDCl₃, 400 MHz): δ 1.30 (s, 3H), 1.31 (s, 3H), 1.71 (m, 1H), 2.12 (m, 1H) 3.49 (ddt, 1H, J=15.0, 9.0, 4.0 Hz), 3.60 (dd, 1H, J=9.0, 6.0 Hz), 4.19 (dd, 1H, J=9.0, 6.0), 4.45 (m, 1H), 4.60 (m, 1H) 7.70 (m, 6H), 7.84 (m, 9H).

### Example 2. Synthesis of Compound 20c

Compound 20c was synthesized as depicted in Scheme 7, according to the following procedure.

Slurry of aluminum chloride (1.79 g, 1.1 eq) in 55 mL DCM was cooled in an ice/water bath. A solution of glutaric anhydride (1.39 g) and 2-penten-4-yn-1-ol 18 (1 g, 12.2 mmol) in 25 mL DCM was added dropwise to the slurry maintaining the temperature. After addition is complete, the reaction is allowed to stir at room temperature overnight. The reaction mixture was added slowly to a 1M HCl solution while maintaining the temperature below 10° C. Mixture was stirred for approximately 45 minutes until a clear solution was observed. The phases were separated, the organic layer washed with brine and dried over sodium sulfate. TLC in 30% ethyl acetate/hexane. Product spot (R_{f}=0.25) was visualized with vanillin and was aqua blue in color. 1 H NMR (CDCl₃, 400 MHz): δ 6.27 (dt, 1H, J=16.0, 6.0 Hz), 5.74 (d, 1H, J=16.0 Hz), 4.63 (d, 2H, 4 Hz), 2.44 (m, 4H), 1.98 (t, 2H, J=8.0 Hz).

Compound 20a (890 mg) was taken up in 25 mL DCM. To the solution were added DIPEA (1.5 mmol, 2 eq) and EtI (0.75 mL). Stir at room temperature overnight and isolated by silica gel column to give compound 20c (1.3 g).

### Example 3. Synthesis of Compound 20b

Dissolve starting material (1.3 g, 5.8 mmol) in 15 mL DCM in an ice/water bath. Imidazole (1 eq) and DMAP (0.05 eq) were added. TBDPSCl (1 eq) was added and the reaction stirred overnight. Reaction was quenched with water and extracted into ether, dried concentrated and chromatographed to give 1.8 g of compound 20b as a white solid.

### Example 4. Synthesis of Compound 26

Compound 26 was synthesized as depicted in Scheme 14, according to the following procedure.

Compound 25 was prepared from 1,2-di-trimethylsilyl acetylene and glutaric anhydride in the presence of aluminum chloride in methylene chloride as described above. Compound 25 (2 g, 9.4 mmol) was dissolved in 25 mL isopropanol, p-TSA (0.1 eq) was added and the reaction mixture stirred at 65° C. overnight. The mixture was concentrated and purified by chromatography to give compound 26 (1.2 g of oil). 1 H NMR (CDCl₃, 400 MHz): δ0.21 (s, 9H), 1.21 (s, 3H), 1.22 (s, 3H), 1.96 (t, 2H, J=6.0 Hz), 2.30 (t, 2H, J=6 Hz), 2.62 (t, 2H, J=8.0 Hz), 4.99 (m, 1H).

### Example 5. Synthesis of Compound 54

Compound 54 was synthesized as depicted in Scheme 17, according to the following procedure.

### TBS Protection of (2R)-1,2-Butane Diol

To a solution of imidazole (1 eq), TBSCl (1 eq), and DMAP (0.05 eq) in 40 mL of DCM at 0-5° C. was added the diol 1 (3 g, 33 mmol). The reaction was stirred and allowed to warm to ambient temperature overnight. The reaction was quenched with ammonium chloride, the product extracted with DCM, the organic layer washed with sodium bicarbonate and brine. Dried over sodium sulfate and concentrated. The material (6.24 g) was carried forward as is. Not UV active, but visible with vanillin (R_{f}=0.5 in 10% ethyl acetate/hexane).

### Compound 51

The material from the previous step (6.24 g, 31.5 mmol) was added to a solution of TBDPSCl (1 eq), imidazole (1 eq), and DMAP (0.05 eq) in DCM at 0-5° C. The reaction was stirred and warmed to ambient temperature overnight. The reaction was quenched with ammonium chloride, the product extracted with DCM, the organic layer washed with sodium bicarbonate and brine. Dried over sodium sulfate and concentrated. The product was purified by column chromatography. Product is UV active at 254 nm. Column eluted with 0-5% ethyl acetate/hexane to give 11.4 g of compound 51 (82% yield).

### Compound 52

The bis-silyl ether 51 (8.3 g, 18.7 mmol) was dissolved in 1:1 DCM:MeOH (50 mL) at rt. CSA (0.5 eq) was added to the reaction mixture. The reaction was stirred for 2 h at rt. Triethylamine (1.1 eq) was added to the reaction mixture to quench. The mixture was concentrated and purified by column chromatography. Product is UV active at 254 nm. Chromatography with 0-20% ethyl acetate/hexane gave 5.34 g of product (87% yield).

### Compound 53

The starting material (1.7 g, 1 eq) was dissolved in 20 mL DCM and TEMPO (0.1 eq) was added. To the stirring reaction mixture was added BAIB (1.2 eq). The reaction was followed by TLC and complete after 3 h. To the reaction mixture was added TEA (2 mL), which was then concentrated and purified by column chromatography (0-20% ethyl acetate/hexane). 1.3 g of product was isolated (77% yield).

### Compound 54

The aldehyde (9.1 g, 27.9 mmol) and (triphenylphosphoranylidene)acetaldehyde (1 eq) were dissolved in 120 mL of chloroform. The reaction was stirred at ambient temperature for 1 h and then refluxed for 2 h. The reaction mixture was concentrated and purified by column chromatography to give 4.9 g of product (50% yield). 1 H NMR (CDCl₃, 400 MHz): δ 0.84 (t, 3H, J=8.0 Hz), 1.08 (s, 9H), 1.51 (m, 2H), 4.43 (m, 1H), 6.17 (dd, 1H, J=16.0, 8.0 Hz), 6.68 (dd, 1H J=14.0, 6.0 Hz), 7.37 (m, 6H), 7.40 (m, 4H), 9.46 (d, 1H, J=8.0 Hz).

### Example 6. Synthesis of Compound 61

Compound 61 was synthesized as depicted in Scheme 19, according to the following procedure.

### Compound 56

The starting alcohol 55 (7 g, 48 mmol) was dissolved in dry DCM (100 mL) and cooled in an ice/water bath. PCC (1.1 eq) was added portion wise over 5 minutes. The reaction mixture was stirred at room temperature for 2 h. The crude mixture was filtered over silica and Celite. The filtrate was carried forward to the next reaction without further manipulation. To the filtrate was added (carbethoxymethylene)triphenylphosphorane (1.1 eq) and the mixture was stirred at room temperature overnight. Following concentration of the reaction mixture and column chromatography (30% ethyl acetate/hexane), 4 g of compound 56 were obtained (40% yield over 2 steps).

### Reduction and Deprotection Reaction of Compound 56

Compound 56 (4 g, 18.7 mmol) was taken up in 30 mL of ethyl acetate at room temperature and a catalytic amount of 10% Pd/C was added. The reaction was stirred under a positive pressure of hydrogen at room temperature for 6 h. The reaction mixture was then filtered over Celite and the filtrate concentrated. The crude material was taken up in 40 mL of 80% AcOH/water and stirred at room temperature overnight. The reaction mixture was concentrated and purified by column chromatography (50-100% ethyl acetate/hexane) to give 2.7 g of diol (82% yield over 2 steps).

### Compound 59

Diol (2.7 g, 15 mmol) was dissolved in 20 mL of DCM. To the solution were added p-Tosyl chloride (1.1 eq), TEA (2 eq) and DMAP (cat). The reaction was stirred at room temperature overnight, concentrated and purified by column chromatography (50% ethyl acetate/hexane) to provide 1.5 g of the tosylate (30% yield).

The tosylate was taken up in 25 mL acetone, and sodium iodide (5 eq) added. The reaction was refluxed for 3 h, cooled, concentrated and purified by column chromatography to yield 1 g of compound 59 (77% yield). 1 H NMR (CDCl₃, 400 MHz): δ −0.14 (s, 3H), −0.11 (s, 3H), 0.79 (s, 9H), 1.25 (t, 3H, J=7.2 Hz), 1.65 (m, 4H), 2.25 (t, 2H, J=7.3 Hz), 3.14 (d, 2H, J=3 Hz), 3.51 (m, 1H), 4.09 (q, 2H, J=7.1 Hz).

### Silylation (61)

The alcohol 59 (1.6 g, 5.6 mmol) was dissolved in 15 mL DCM. Imidazole (1 eq) and DMAP (cat) were added and the reaction mixture cooled in an ice/water bath. To the cooled reaction, TBSCl (1 eq) was added. The reaction was stirred at room temperature overnight. The reaction was quenched with saturated aqueous ammonium chloride and diluted with 20 mL DCM. The organic phase was washed with saturated sodium bicarbonate solution and brine, dried over sodium sulfate, filtered, concentrated and purified by column chromatography to yield 1.3 g (58% yield).

### Acetylation (Alternate to Silyl Protection)

In an alternative route not shown in the Scheme, the alcohol 59 (1.0 g, 3.5 mmol) was dissolved in 25 mL DCM. TEA (2 eq) and DMAP (cat) were added and the reaction mixture cooled in an ice/water bath. To the cooled reaction, acetic anhydride (1.25 eq) was added. The reaction was stirred at room temperature overnight. The reaction was concentrated and purified by column chromatography to give 820 mg of acetate (75% yield)

### Formation of Salt 61

The iodide (3.25 mmol) was dissolved in 25 mL acetonitrile, and triphenylphosphine (1.5 eq) and sodium bicarbonate (1 eq) were added. The reaction mixture was refluxed for 2 d, cooled, filtered and the filtrate concentrated and purified by column chromatography to give 700 mg (33% yield) of salt 61 (with PG=TBDMS). 1 H NMR (CDCl₃, 400 MHz): δ 1.30 (t, 3H, J=7.5 Hz), 1.53 (m, 2H), 1.65 (m, 2H), 2.10 (s, 3H), 2.35 (m, 3H), 3.74 (m, 1H) 4.16 (q, 2H, J=7.3 Hz), 4.93 (m, 1H), 7.45 (m, 15H).

Formation of Wittig Salt with Acetate

The salt 61 (with PG=OAc) was formed using the same procedure as above. 40% yield.

### Example 7. Synthesis of Compound 70

Compound 70 was synthesized as depicted in Scheme 18, according to the following procedure.

### Compound 64

To a solution of the alcohol (5 g) in 20 mL DCM in an ice/water bath was added TEA (2 eq) p-Tosyl chloride (1.1 eq), and DMAP (cat). The reaction was warmed to room temperature and stirred for 1.5 h. The reaction mixture was concentrated and purified by column chromatography (30% ethyl acetate/hexane) to give 10 g of tosylate (quantitative). The tosylate was taken up in 30 mL of acetone and sodium iodide (1.5 eq) added. The reaction mixture was refluxed for 2.5 h, cooled and quenched with water. After extraction into ethyl acetate, drying, filtering, concentrating and purification via column chromatography 6.2 g of iodide 64 were obtained (71% yield from tosylate).

Preparation of Wittig Salt from 64

The iodide (19 g, 74.2 mmol), triphenylphosphine (1.2 eq) and sodium bicarbonate (1 eq) were suspended in 40 mL acetonitrile and the mixture refluxed for 2 d. The reaction mixture was cooled to room temperature, filtered through Celite and washed with 100 mL DCM. The filtrate was concentrated and the residue was treated with ether to give a white solid which was collected by filtration and dried to give 35 g of salt (91%).

Preparation of Diol from Wittig Salt

The salt was taken up in 75 mL of 80% AcOH/water and stirred at ambient temperature overnight. The reaction mixture was concentrated and after column chromatography 11.2 of diol were obtained (quantitative yield).

Preparation of Diol from Iodide

Iodide 64 was taken up in 50 mL of 80% AcOH/water and stirred at room temperature for 2 h. After concentration and column chromatography (75-100% ethyl acetate) to yield 2.3 g of diol (44%). 1 H NMR (CDCl₃, 400 MHz): δ 1.96 (td, 2H, J=7.6, 6.1 Hz), 3.3 (t, 2H, J=7.5 Hz), 3.47 (d, 2H, J=4 Hz), 3.80 (tt, 1H, J=6.5 Hz)

Preparation of 66 (di-TBS)

The diol (6 g, 27.8 mmol) was dissolved in 60 mL DCM in an ice/water bath. Imidazole (2.2 eq), TBSCl (2.2 eq) and DMAP (0.04 eq) were added and the reaction stirred at room temperature overnight. The reaction mixture was quenched with saturated ammonium chloride and diluted with DCM. The organic phase was washed with saturated sodium bicarbonate solution, brine and dried filtered, concentrated and purified by column chromatography to yield 9.9 g (80% yield).

### Mono Deprotection of 66

The bis-silyl ether (300 mg) was taken up in 5 mL 80% AcOH/water, 0.5 mL MeOH and stirred at ambient temperature overnight. After concentration and chromatography 125 mg of primary alcohol was obtained. 1 H NMR (CDCl₃, 400 MHz): δ 0.12 (s, 3H), 0.14 (s, 3H), 0.91 (s, 9H), 2.05 (m, 2H), 3.21 (m, 2H), 3.49 (m, 1H), 3.61 (m, 1H), 3.86 (m, 1H).

### Preparation of Di-TBS Wittig Salt

Salt was prepared from the iodide via the usual procedure. After column chromatography the salt was obtained in 40% yield.

Wittig Reaction of Di-TBS Wittig Salt with 54

Reaction was performed according to the same procedure as the synthesis of 71 (see Example 8). 1 H NMR (CDCl₃, 400 MHz): δ 0.00 (m, 12H), 0.79 (d, 3H, J=7 Hz) 0.86 (s, 9H), 0.89 (s, 9H), 1.14 (s, 9H), 1.5 (m, 2H), 2.17 (m, 1H), 2.25 (m, 1H), 3.40 (dd, 1H, J=12, 8 Hz), 3.48 (m, 1H), 3.67 (m, 1H), 4.12 (m, 1H), 5.39 (m, 1H), 5.57 (dd, 1H, J=16, 8 Hz), 5.93 (t, 1H, J=9 Hz), 6.10 (dd, 1H, J=16, 9 Hz), 7.40 (m, 6H), 7.65 (m, 4H).

### Preparation of OTBS/OTBDPS (65)

Imidazole (1 eq) and DMAP (cat) were dissolved in 35 mL DCM in an ice/water bath and stirred for 5 min. TBSCl (1 eq) was added to the mixture and it was stirred an additional 5 min. The diol (2.3 g, 10.6 mmol) in 18 mL DCM was added and the reaction was stirred at room temperature overnight. The reaction mixture was quenched with saturated ammonium chloride and diluted with DCM. The organic phase was washed with saturated sodium bicarbonate solution, brine and dried filtered, concentrated and purified by column chromatography to yield 2.7 g (82% yield). The product was dissolved in 20 mL DCM and cooled in an ice/water bath. To the solution was added imidazole (1 eq), and DMAP (cat), after stirring 5 min the TBDPSCl (1 eq) was added and the reaction mixture stirred at room temperature overnight. The reaction was worked up as before and chromatographed to yield 4.2 g of bis-silyl ether (90%).

### CSA Deprotection of OTBS/OTBDPS

The bis-silyl ether (4.2 g, 7.45 mmol) was dissolved in 1:1 MeOH:DCM (30 mL) and CSA (0.5 eq) was added. The mixture was stirred at room temperature for 2 h. TEA (5 mL) was added and the reaction mixture concentrated. After column chromatography 1.2 g of alcohol were obtained (36%).

### Preparation of OTBDPS Aldehyde (68)

The alcohol (1.2 g) was dissolved in 15 mL DCM and cooled in an ice/water bath. DMP (1.1 eq) was added and the mixture stirred at room temperature for 3 h. The mixture was diluted with DCM (50 mL) and then washed with a 1:1 mixture of NaHCO₃ and Na₂S₂O₃ aqueous solution, saturated sodium bicarbonate solution, and brine. Upon drying and concentration the crude material was purified by column chromatography to give 725 mg of aldehyde (60% yield).

### Preparation of Aldehyde 68

The OTBDPS aldehyde (1.38 g, 3.1 mmol) was dissolved in 20 mL DCM and (triphenylphosphoranylidene)acetaldehyde (1.3 eq) added. The mixture was stirred at room temperature overnight. Hexane (30 mL) was added to the reaction mixture, which was filtered through Celite and the filter pad washed with hexane. The filtrate was concentrated and purified by column chromatography to give 608 mg (40% yield). 1 H NMR (CDCl₃, 400 MHz): δ 1.12 (s, 9H), 2.05 (m, 2H), 3.10 (m, 2H), 4.56 (dt, 1H, J=7.4, 7.3 Hz), 6.12 (dd, 1H, J=16, 8 Hz), 6.60 (dd, 1H, J=16, 8 Hz), 7.61 (m, 5H), 9.40 (d, 1H, J=8 Hz).

### Preparation of OTBDPS Amide (69)

The compound 68 (600 mg, 1.3 mmol) and the Wittig salt Ph₃P═CHCON(OMe)Me (2 eq) were dissolved in 20 mL DCM and stirred at ambient temperature overnight. Upon concentration and column chromatography 415 mg of the E-isomer and 120 mg of the Z-isomer were obtained (73% yield overall). The E-isomer 69 was carried forward to the next step.

Salt Preparation from the OTBDPS Amide (70)

The iodide 69 (415 mg, 0.73 mmol) was dissolved in 15 mL of acetonitrile. Triphenylphosphine (1.2 eq) and sodium bicarbonate (1.2 eq) were added and the reaction refluxed for 3 d. Upon concentration and column chromatography 549 mg of the salt were obtained (91% yield).

### Example 8. Synthesis of Compound 72

Compound 72 was synthesized as depicted in Scheme 16, according to the following procedure.

### Compound 71

The salt 70 (186 mg, 0.23 mmol) was dissolved in 5 mL dry THF and cooled to −78° C. and stirred for 15 minutes, and then KHMDS (0.5M in toluene) (1.5 eq) was added. Stirred for 30 min at −78° C. and 30 min at ambient temperature. HMPA (2 mL) was added and the reaction cooled to −78° C. and 54 (1.2 eq) was added. The reaction was stirred for 1 h and then warmed to ambient temperature and stirred an additional 30 min. The reaction was quenched with water and extracted into ethyl acetate. Upon drying and concentrating the organic layer, column chromatography yielded 36 mg (24% yield).

### Compound 72

The preparation of compound 72 is carried out by DIBAL reaction from compound 71. In particular, 36 mg of compound 71 was stirred in 3 mL THF and cooled to −78° C. 3 eq of DIBALH added and stirred for 2 h. The reaction mixture was split between water and ethyl acetate. Filtration and concentration followed by column chromatography gave 35 mg of the desired aldehyde.

### Example 9. Synthesis of RvE1 Salt (Compound 30)

The sodium salt of RvE1 (compound 30) was synthesized as depicted in Scheme 15, according to the following procedure.

### Preparation of OTBS/OTBDPS Salt

The OTBS/OTBDPS iodide (1.5 g) was taken up in acetonitrile. Triphenylphosphine (1.2 eq) and sodium bicarbonate (1.2 eq) were added and the reaction refluxed for 3 d. Upon concentration and column chromatography 946 mg of the salt were obtained (44% yield).

### Wittig Reaction of OTBS/OTBDPS Salt and 54

The salt (1.31 g, 1.57 mmol) was dissolved in 10 mL dry THF, cooled to −78° C. and stirred for 5 min KHMDS (0.5M/toluene) (1 eq) was slowly added to the solution. The orange solution was stirred at −78° C. for 15 min and 54 (1 eq) in 5 mL THF was added over 2 minutes. The reaction temperature was maintained for 10 min and then warmed to room temperature for 10 min. The reaction was quenched with ice water and the THF removed on the rotovap. The aqueous was extracted with ethyl acetate, dried and concentrated. The crude was purified by column chromatography to give 845 mg product (70%). 1 H NMR (CDCl₃, 400 MHz): δ −0.14 (s, 3H), −0.11 (s, 3H), 0.79 (s, 12H), 1.03 (m, 18H), 1.50 (m, 2H), 2.25 (m, 2H), 3.40 (m, 2H), 3.75 (m, 1H), 4.07 (m, 1H), 5.37 (dd, 1H, J=16, 8 Hz), 5.53 (dd, 1H, J=16, 8 Hz), 5.89 (m, 2H), 7.34 (m, 12H), 7.65 (m, 8H).

### Wittig Reaction of Compound 72 and Compound 61

RvE1 could be made from compounds 72 and 61 using NaH, KHMDS, NaHMDS, nBuLi, LDA, K₂CO₃, NA₂CO₃ in THF, toluene, DMF, ether, di-tert-butyl ether at −78° C. to rt.

Basically, 61 will be dissolved in one of these solvents and cooled. Base will be added and after 1-2 hr the aldehyde 72 will be added at −78° C., and will be stirred up to rt to get the coupling product. Silyl groups will be deprotected using TBAF and ammonium chloride in THF, followed by LiOH or NaOH hydrolysis in EtOH or MeOH or THF to obtain RvE1 salt.

### APPENDIX

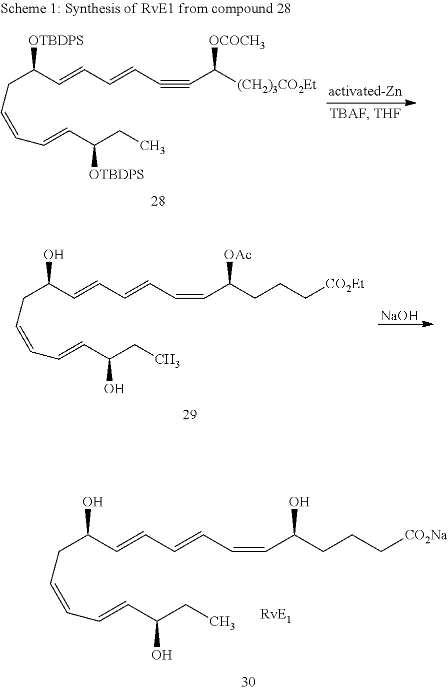

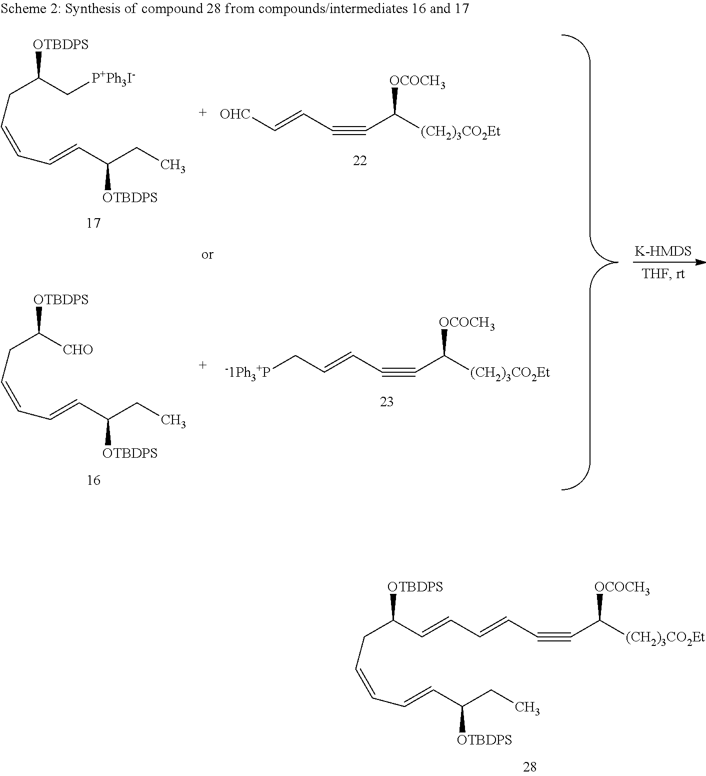

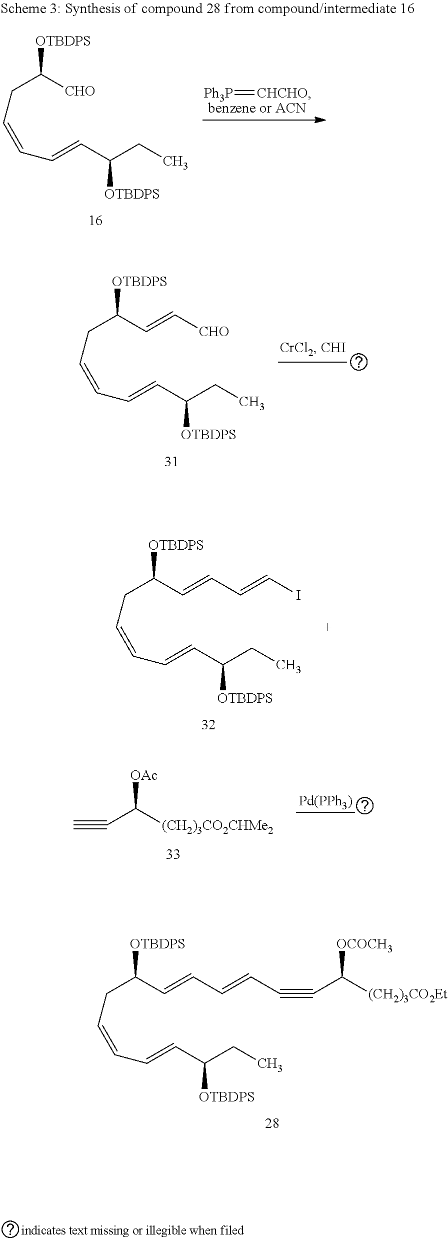

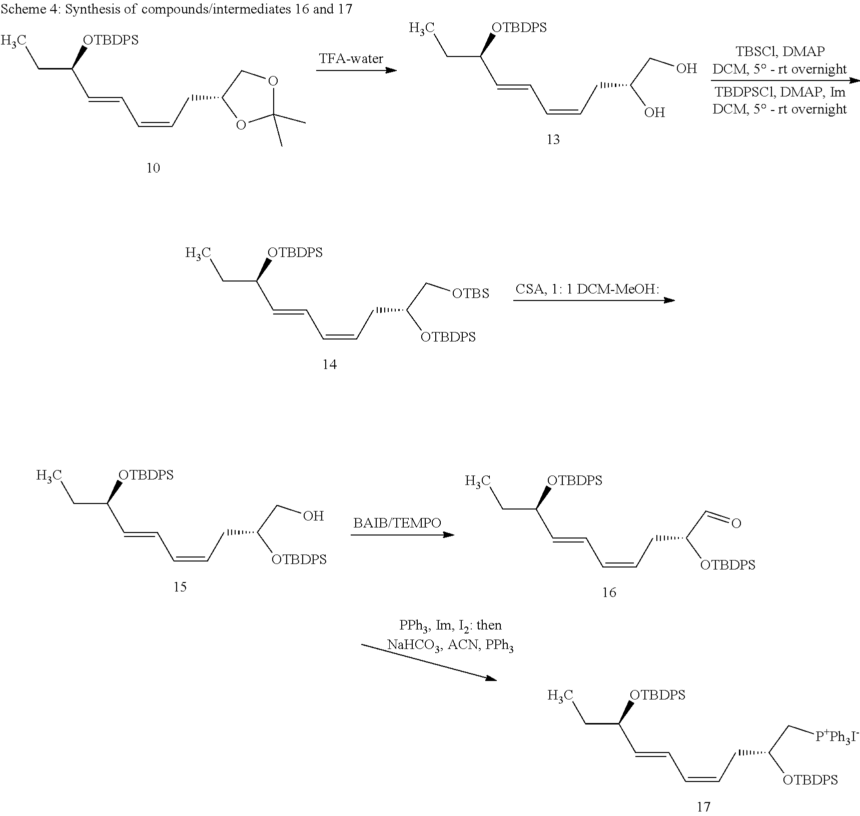

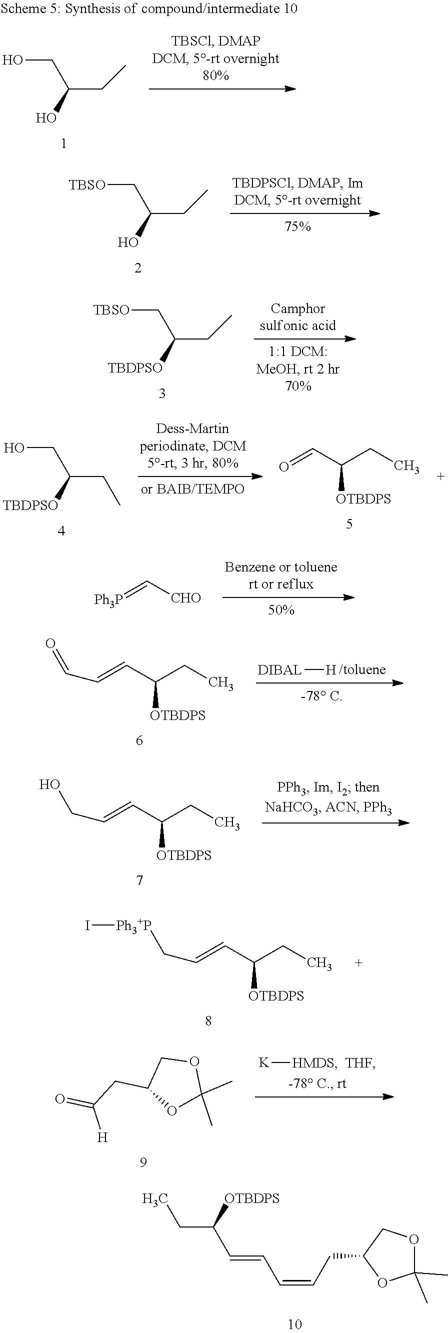

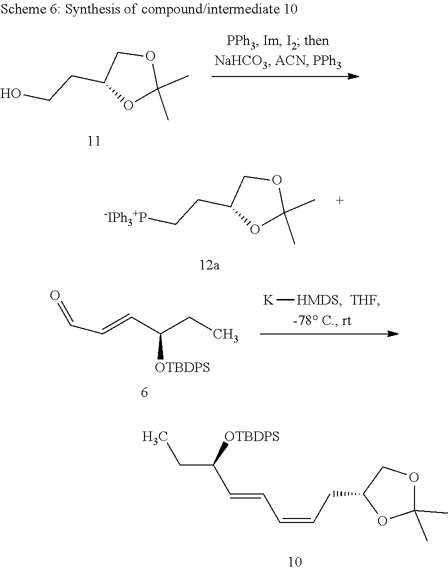

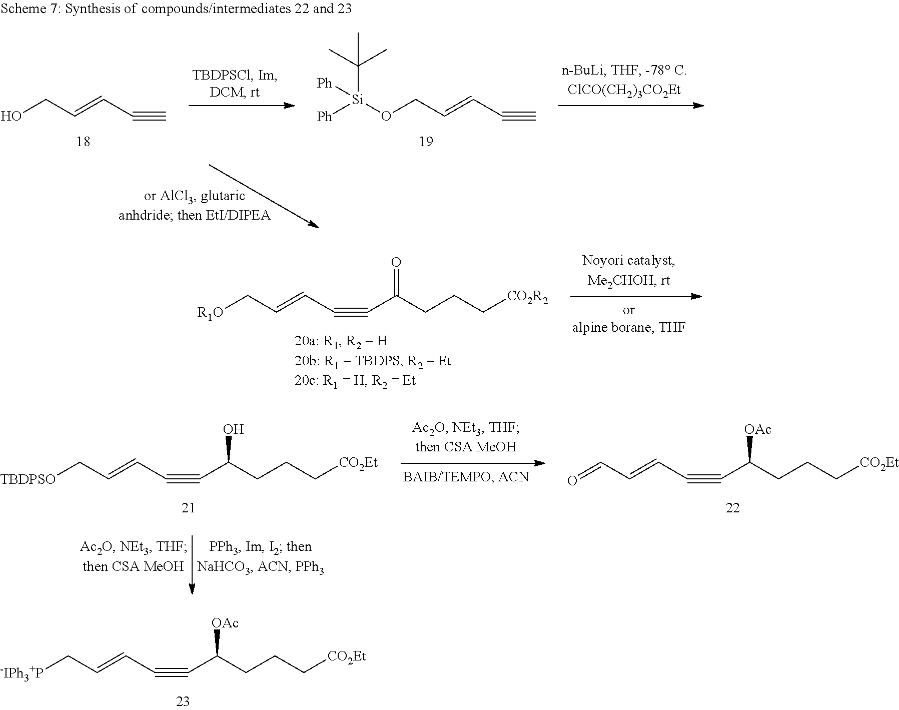

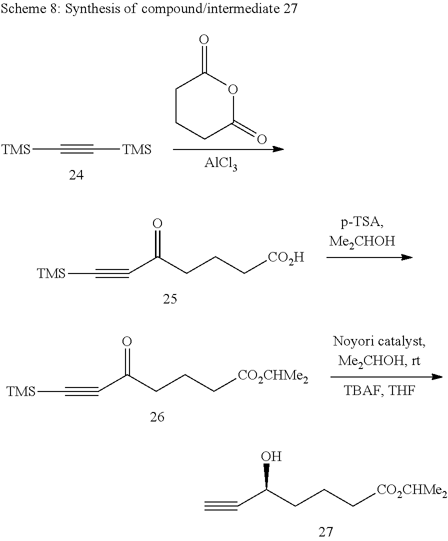

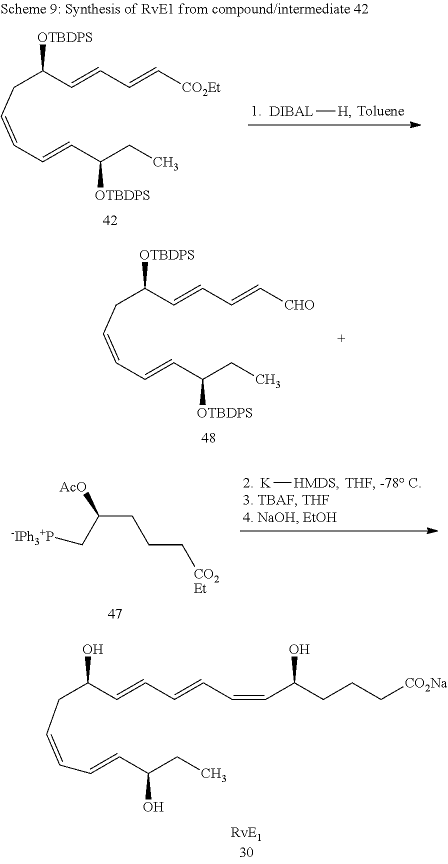

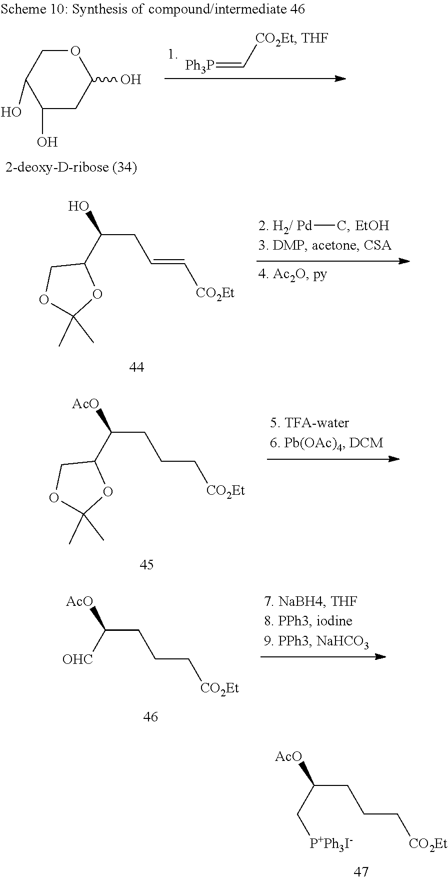

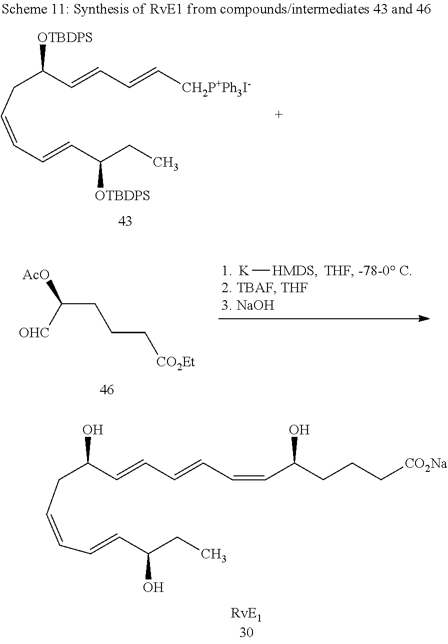

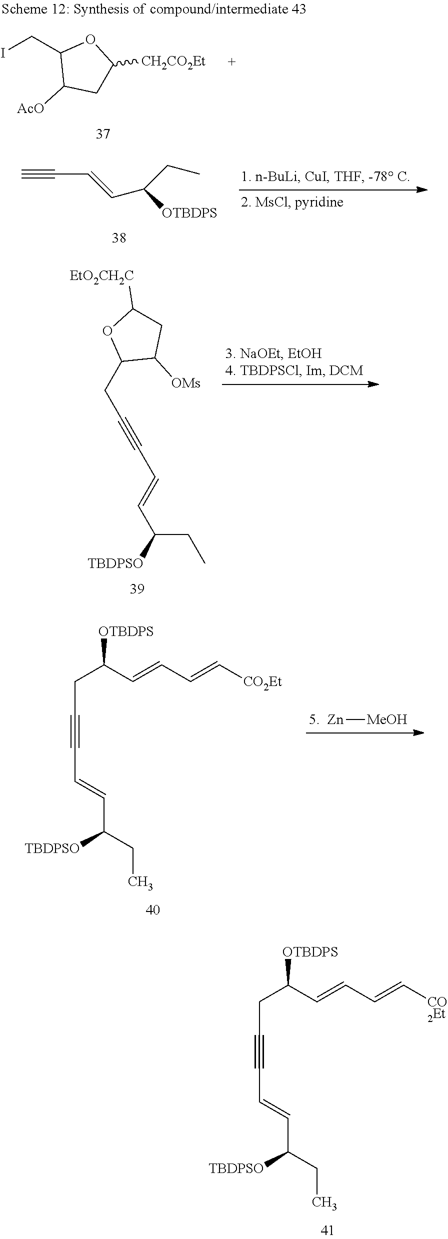

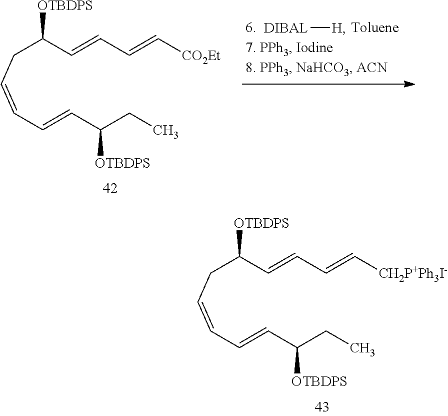

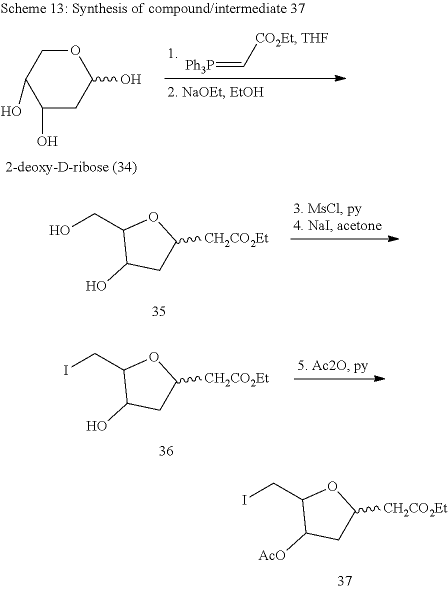

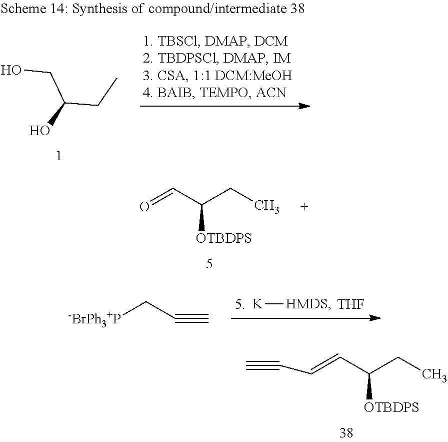

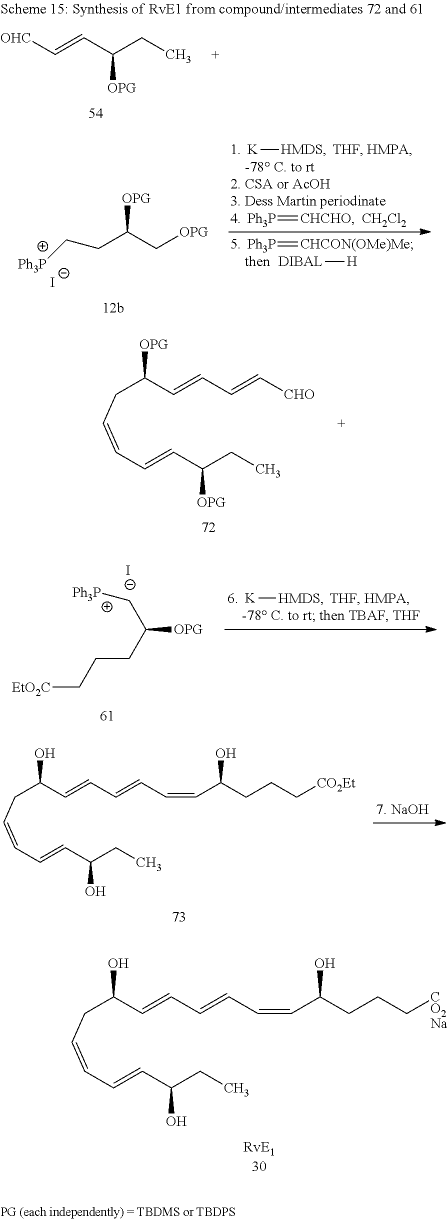

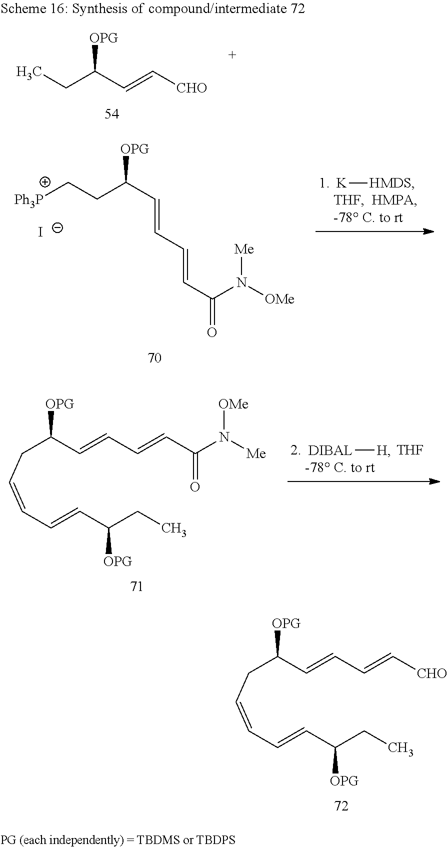

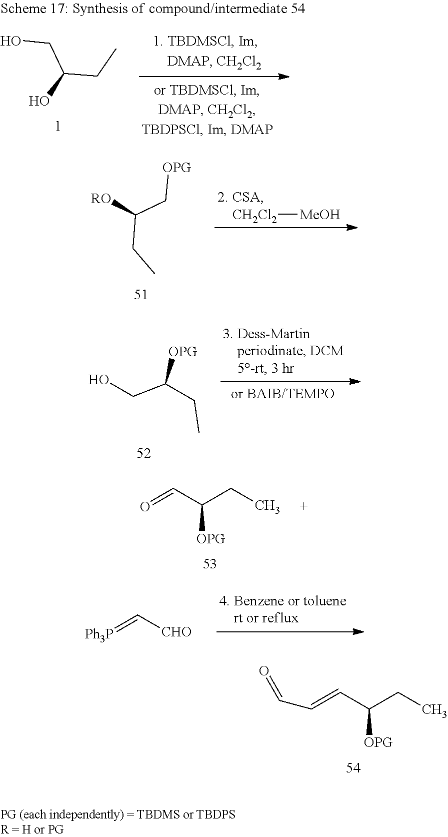

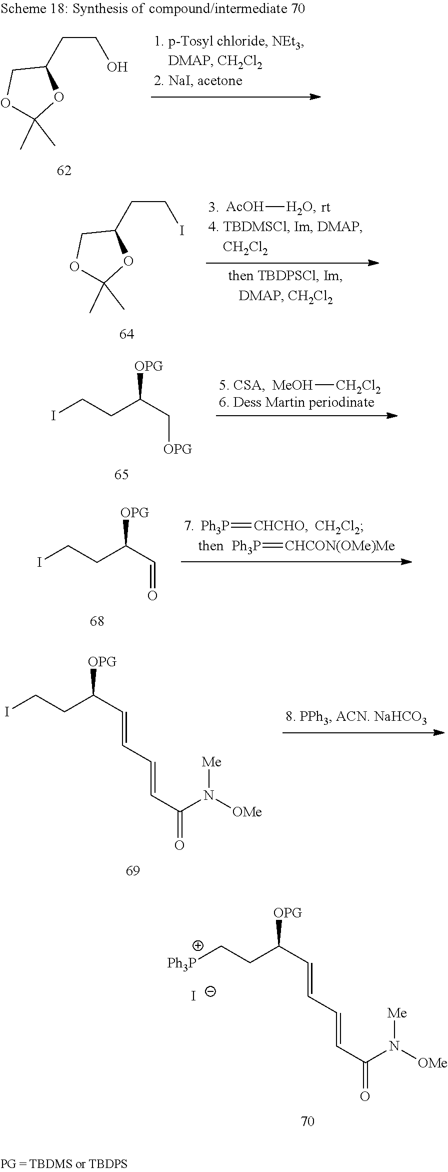

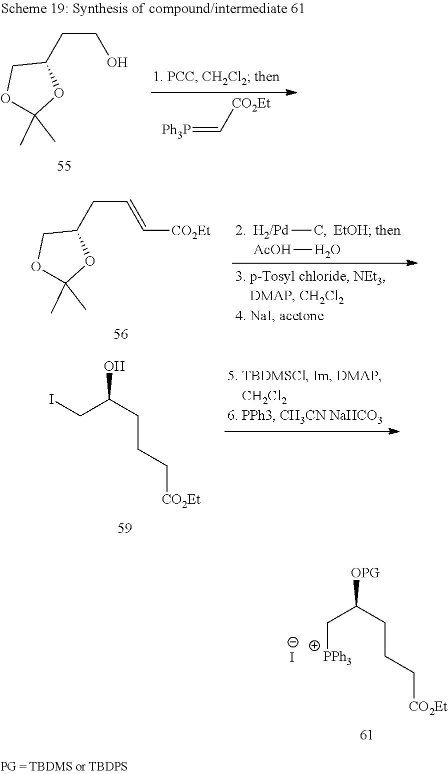

1. A method for the synthesis of Resolvin E1 (RvE1) of the formula 30 starting from compounds 72 and 61, wherein PG each independently is a hydroxyl-protecting group, said method is carried out as depicted in Scheme 15 and comprises: (i) Wittig reaction of compound 72 with compound 61 in the presence of a strong base; (ii) removal of the hydroxyl-protecting groups with a deprotecting reagent to obtain compound 73; and (iii) hydrolysis of the ester group of compound 73, to obtain RvE1.

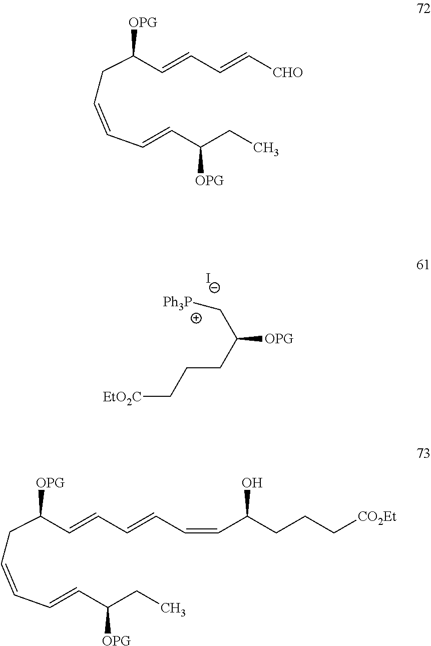

2. The method of claim 1, wherein said compound 72 is synthesized as depicted in Scheme 16 by (i) Wittig reaction of compound 54 and compound 70, wherein PG each independently is a hydroxyl-protecting group, in the presence of a strong base to obtain compound 71; and (ii) removal of the amide group of compound 71 with a strong base to obtain compound 72.

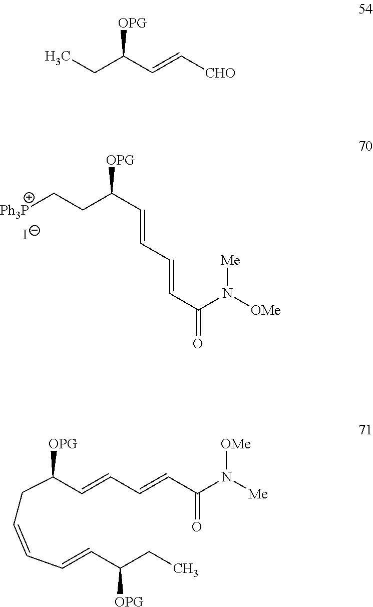

3. The method of claim 2, wherein said compound 54 is synthesized as depicted in Scheme 17 by Wittig reaction of compound 53, wherein PG is a hydroxyl-protecting group, and Ph₃P═CHCHO.

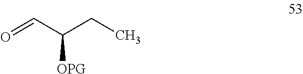

4. The method of claim 2, wherein said compound 70 is synthesized starting from compound 64, said method is carried out as depicted in Scheme 18 and comprises: (i) deprotection of the diol in the presence of a weak acid; (ii) protection of the hydroxyl groups to obtain compound 65, wherein PG each independently is a hydroxyl-protecting group; (iii) Dess-Martin oxidation of compound 65 with Des s-Martin periodinate to obtain aldehyde 68; (iv) double Wittig reaction of aldehyde 68 with Ph₃P═CHCHO and then with Ph₃P═CHCON(OMe)Me to obtain compound 69; and (v) conversion of said compound 69 to the triphenylphosphonium salt 70 with triphenylphosphine.

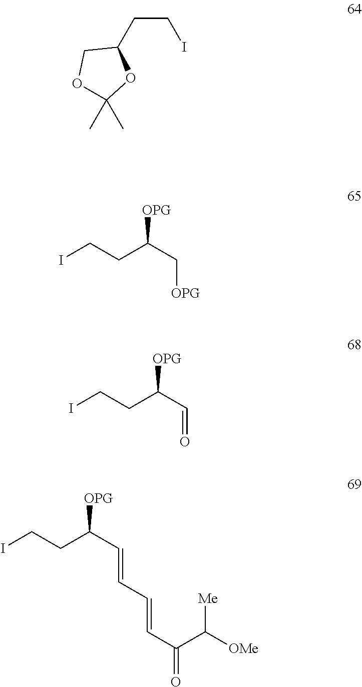

5. The method of claim 1, wherein said hydroxyl-protecting group is tert-butyldimethylsilyl ether (TBDMS) or tert-butyldiphenylsilyl ether (TBDPS).

6. The method of claim 1, wherein said compound 61 is synthesized starting from compound 56, said method is carried out as depicted in Scheme 19 and comprises: (i) reduction and deprotection of compound 56, followed by tosylation and then iodination to obtain compound 59; and (ii) hydroxyl-protection of compound 59 followed by conversion to the triphenylphosphonium salt 61 with triphenylphosphine.

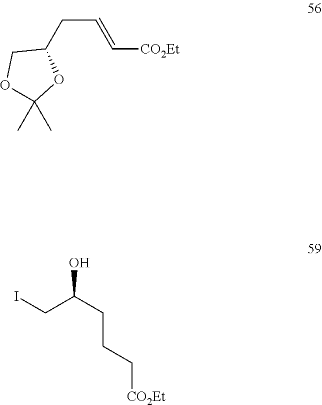

7-14. (canceled)

15. A method for the synthesis of Resolvin E1 (RvE1) of the formula 30 starting from compound 42, said method is carried out as depicted in Scheme 9 and comprises: (i) reduction of the ester group of compound 42 to obtain compound 48; (ii) Wittig reaction of compound 48 with compound 47 in the presence of a strong base to obtain an intermediate product: and (iii) removal of the hydroxyl-protecting groups at positions C12 and C18 of said intermediate product and deacetylation of the protected hydroxyl group at position C5 of said intermediate product, to obtain RvE1.

16. The method of claim 15, wherein compound 47 used in the synthesis is synthesized starting from 2-deoxy D-ribose (compound 34) as depicted in Scheme 10.

17. A method for the synthesis of Resolvin E1 (RvE1) of the formula 30 starting from compounds 43 and 46, said method is carried out as depicted in Scheme 11 and comprises: (i) Wittig reaction of compound 43 with compound 46 in the presence of a strong base to obtain an intermediate product; and (ii) removal of the hydroxyl-protecting groups at positions C12 and C18 of said intermediate product and deacetylation of the protected hydroxyl group at position C5 of said intermediate product, to obtain RvE1.

18. The method of claim 17, wherein said compound 43 is synthesized starting from compounds 37 and 38 as depicted in Scheme 12.

19. The method of claim 17, wherein said compound 46 is synthesized starting from 2-deoxy D-ribose (compound 34) as depicted in Scheme 10.

20. The method of claim 18, wherein said compound 37 is synthesized starting from 2-deoxy D-ribose (compound 34) as depicted in Scheme 13.

21. The method of claim 18, wherein said compound 38 is synthesized starting from compound 1 as depicted in Scheme 14.

22. A compound selected from the group consisting of compounds 38, 39, 40, 41, 42, 47, 59, 61, 65, 68, 69, 70, 71, 72, and 73 shown below: 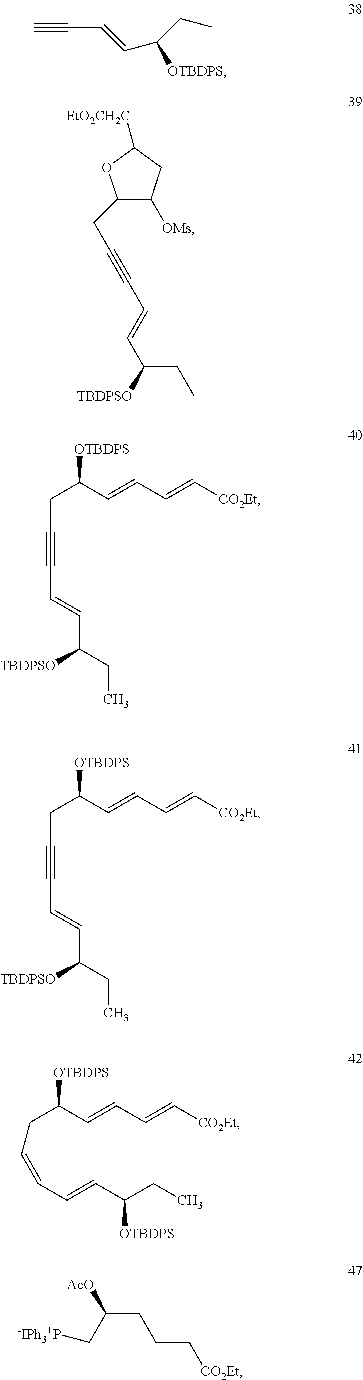 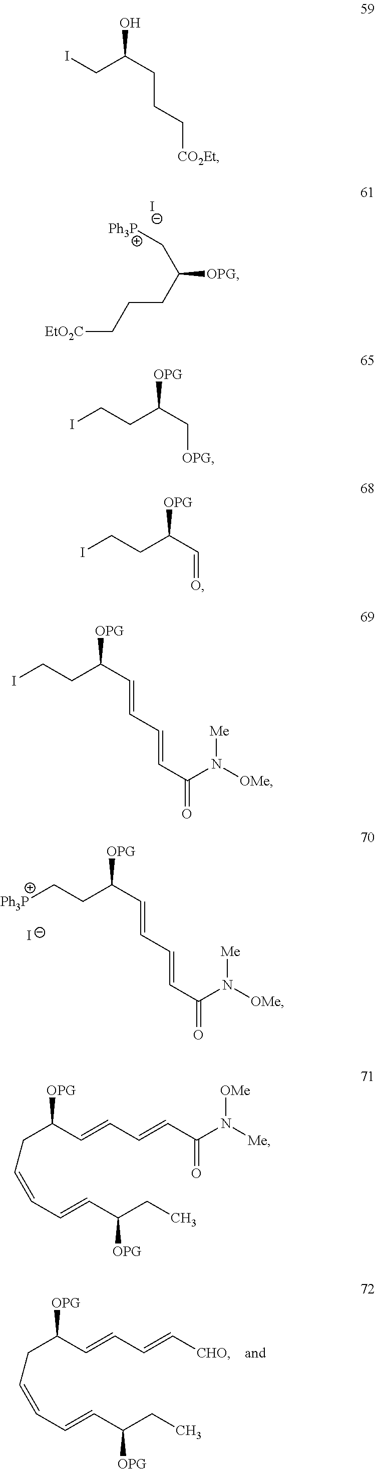 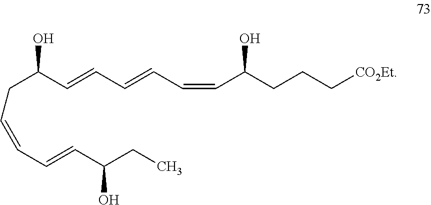 [SMILES: CCOC(=O)CCC[C@H](O)\C=C/C=C/C=C/[C@H](O)C/C=C\C=C\[C@H](O)CC]
